(19) United States
(12) Patent Application Publication
(10) Pub. No.: US 2024/0389917 A1
(43) Pub. Date: Nov. 28, 2024

(54) Response Profile Evaluation Device, Response Profile Evaluation Method, and Response Profile Evaluation Program

(71) Applicant: Hitachi, Ltd., Tokyo (JP)

(72) Inventor: Shunsuke MINUSA, Tokyo (JP)

(21) Appl. No.: 18/606,643

(22) Filed: Mar. 15, 2024

(30) Foreign Application Priority Data
May 26, 2023 (JP) 2023-087058

(51) Int. Cl. A61B 5/16; G16H 50/30

(52) U.S. Cl. CPC A61B 5/165; G16H 50/30

(57) ABSTRACT

A response profile evaluation device of the present invention can quantitively evaluate a response profile occurring in rating value data. A response profile evaluation device of the present invention, using a response tendency of a user as a response profile, and using a statistical distribution expressing the response profile of the user as a response profile distribution, includes: a receiving unit for receiving rating value data of the user; a response profile distribution estimating unit for estimating, based on the rating value data, response profile distribution candidates that are candidates of the response profile distribution, by using a candidate response profile distribution template including a type of the statistical distribution; and a response profile distribution evaluating unit for selecting the response profile distribution that is fitted to expression of the response profile of the user from the estimated response profile distribution candidates by using the rating value data.

### CLAIM OF PRIORITY

The present application claims priority from Japanese Patent Application JP 2023-087058 filed on May 26, 2023, the content of which is hereby incorporated by reference into this application.

### BACKGROUND OF THE INVENTION

The present invention relates to a response profile evaluation device, a response profile evaluation method, and a response profile evaluation program used at the time of measuring and evaluating a subjective health condition of the user.

As means for obtaining the subjective health condition in daily life with time and easily, psychological measurement using measurement scales is employed. As the measurement scales, for example, the Likert scale as an ordinal scale of a multivalued discrete response type or the like is generally used. In recent years, VAS (Visual Analogue Scale) measurement using, as a measurement scale, the visual analogue scale (VAS) as an interval scale of a continuous response type is employed for psychological measurement. When the VAS is used as a measurement scale, responses can be obtained from subjects more easily and with higher granularity. Data of a response of a subject (user) to an instruction will be called rating value data.

In the psychological measurement using the measurement scale, a response tendency which is consistent of each subject and occurs regardless of an instruction may occur. The response tendency is included in rating value data and can exert adverse influence on, particularly, intersubject analysis using a psychological measurement value.

An example of a technique of removing such a response tendency which occurs in a subject is disclosed in document JP 2021-026647. The document JP 2021-026647 discloses a response style component eliminating device capable of eliminating a response style (RS) as a response tendency which is independent of the content of questionnaire, targeting a response tendency appearing at the time of evaluation of subjective health condition by using the Likert scale.

As described above, in recent years, VAS measurement using, as a measurement scale, a VAS as an interval scale of a continuous response type is employed. Consequently, also in VAS measurement in which the measurement scale is continuous values, a technique capable of evaluating a response tendency quantitively and eliminating the response tendency is in demand. Hereinafter, the response tendency will be also called a response profile (RP).

In the document JP 2021-026647, it is described that the device in JP 2021-026647 can be used also in the case where the measurement scale is made of continuous values. However, the nature of a response of subjective assessment in an interval scale (for example, VAS) is different from that of a response of subjective assessment in an ordinal scale (for example, Likert scale), it is generally difficult to apply the technique of eliminating a response profile in the case of using an ordinal scale such as the technique described in JP 2021-026647 to the case of using an interval scale such as a VAS. Particularly, a VAS often includes items of different types like in a unipolar VAS and a bipolar VAS, and has a problem such that evaluation is difficult by a method premised on a single mathematical model like a conventional technique.

### SUMMARY OF THE INVENTION

An object of the present invention is to provide a response profile evaluation device, a response profile evaluation method, and a response profile evaluation program which are capable of quantitively evaluating a response profile occurring in rating value data.

A response profile evaluation device according to the present invention, which uses a response tendency of a user as a response profile, and uses a statistical distribution expressing the response profile of the user as a response profile distribution, comprises: a receiving unit for receiving rating value data of the user; a response profile distribution estimating unit for estimating, on the basis of the rating value data, a plurality of response profile distribution candidates that are candidates of the response profile distribution, by using a candidate response profile distribution template including a type of the statistical distribution; and a response profile distribution evaluating unit for selecting the response profile distribution that is fitted to expression of the response profile of the user from the plurality of estimated response profile distribution candidates by using the rating value data.

A response profile evaluation method according to the present invention, executed by a computer comprising a receiving unit, a response profile distribution estimating unit, and a response profile distribution evaluating unit, and using a response tendency of a user as a response profile, and using a statistical distribution expressing the response profile of the user as a response profile distribution, comprising: a step by the receiving unit, of receiving rating value data of the user; a step by the response profile distribution estimating unit, of estimating, on the basis of the rating value data, a plurality of response profile distribution candidates that are candidates of the response profile distribution, by using a candidate response profile distribution template including a type of the statistical distribution; and a step by the response profile distribution evaluating unit, of selecting the response profile distribution that is fitted to expression of the response profile of the user from the plurality of estimated response profile distribution candidates by using the rating value data.

A response profile evaluation program according to the present invention is a program for making a computer have functions of the receiving unit, the response profile distribution estimating unit, and the response profile distribution evaluating unit of the response profile evaluation device according to the present invention.

### Advantageous Effects of Invention

The present invention provides the response profile evaluation device, the response profile evaluation method, and the response profile evaluation program capable of quantitively evaluating a response profile occurring in rating value data.

### BRIEF DESCRIPTION OF THE DRAWINGS

FIG. 1 is a block diagram illustrating an example of main components of a response profile evaluation device according to a first embodiment of the present invention;

FIG. 2 is a flowchart illustrating an example of process of receiving data from a user terminal, by the response profile evaluation device;

FIG. 3 is a flowchart illustrating an example of process of estimating a response profile distribution (RP distribution) executed by the response profile evaluation device;

FIG. 4 is a flowchart illustrating an example of RP distribution evaluating process performed by the response profile evaluation device;

FIG. 5 is a result of an experiment of RP distribution estimation and RP distribution evaluation performed by using the response profile evaluation device of the embodiment;

FIG. 6 is a diagram illustrating an example of RP distribution data of the user, which is displayed on a user terminal or an administrator terminal;

FIG. 7 is a diagram illustrating an example of a data structure of RP distribution data;

FIG. 8 is a flowchart illustrating an example of a series of processes of evaluating an RP distribution with a confidence interval in a second embodiment of the present invention, which are performed by a response profile evaluation device;

FIG. 9 is a diagram illustrating an example of RP distribution data with a confidence interval of the user, which is displayed on a user terminal or an administrator terminal;

FIG. 10 is a block diagram illustrating an example of main components of a response profile evaluation device according to a third embodiment of the present invention;

FIG. 11A is a flowchart illustrating an example of processes of leaning a subjective condition evaluation model, which are performed by the response profile evaluation device;

FIG. 11B is a diagram illustrating an example of processes of subjective condition evaluation, which are performed by the response profile evaluation device;

FIG. 12 is a block diagram illustrating an example of main components of a response profile evaluation device according to a fourth embodiment of the present invention;

FIG. 13A is a flowchart illustrating an example of processes of receiving data from a user terminal, by the response profile evaluation device;

FIG. 13B is a flowchart illustrating an example of processes of learning a biological condition evaluation model, performed by the response profile evaluation device;

FIG. 13C is a diagram illustrating an example of processes of biological condition evaluation, performed by the response profile evaluation device;

FIG. 14A is a diagram illustrating an example of an evaluation result displayed on a user terminal by a result displaying unit, as an example of a display screen at the time of requesting the user to enter rating value data on the basis of evaluation result data; and

FIG. 14B is a diagram illustrating an example of an evaluation result displayed on the user terminal by the result displaying unit, as an example of the display screen displayed to the user of an example of a plurality of RP types A to D and the RP type of the user.

### DETAILED DESCRIPTION OF THE PREFERRED EMBODIMENTS

According to the present invention, rating value data, particularly, a response profile (response tendency of the user) occurring in rating value data measured by using a measurement scale of a continuous response type such as a VAS can be quantitively evaluated. By obtaining a response profile, an adverse influence of the response profile exerted on an intersubject analysis can be eliminated. In the present invention, also for rating value data measured by using a measurement scale of a continuous response type such as a VAS, in which items of different types of measurement scales such as a unipolar scale and a bipolar scale are often included, a response profile (RP) can be quantitively evaluated in a form that can be compared with other users. It leads to realization of an intersubject analysis of a psychometric value in which the influence of the RP is considered.

A response profile evaluation method according to the present invention can be executed by a response profile evaluation device according to the present invention. A response profile evaluation program according to the present invention is a program for making a computer realize functions of function units of a response profile evaluation device according to the present invention.

Hereinafter, a response profile evaluation device, a response profile evaluation method, and a response profile evaluation program according to embodiments of the present invention will be described with reference to the drawings. In the following description, a response profile as a response tendency of the user will be also described as “RP”.

### First Embodiment

A response profile evaluation device, a response profile evaluation method, and a response profile evaluation program according to a first embodiment of the present invention will be described.

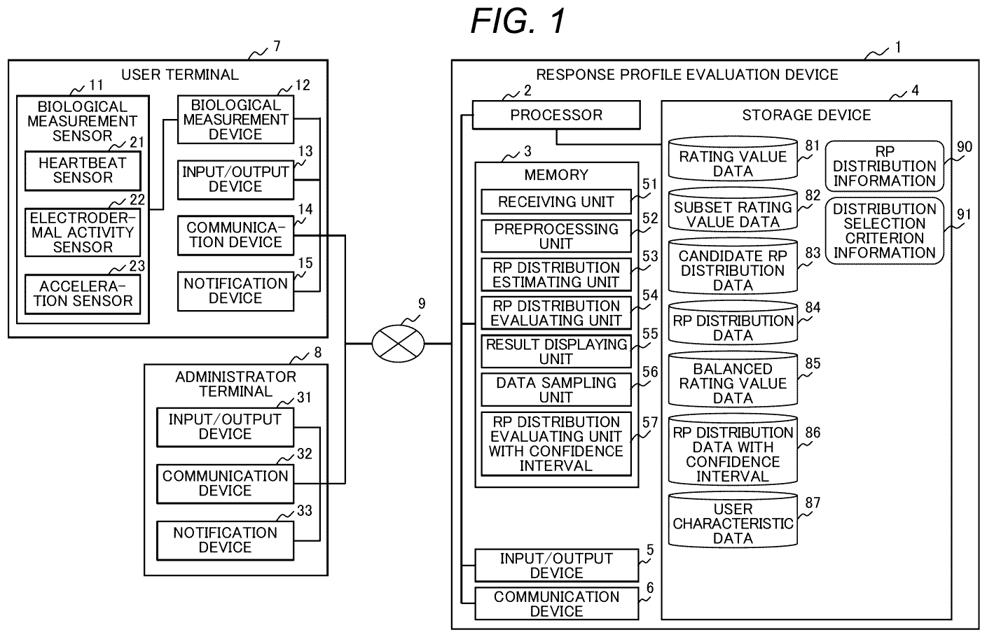

FIG. 1 is a block diagram illustrating an example of main components of a response profile evaluation device 1 according to the embodiment. A response profile evaluation device system is configured by the response profile evaluation device 1 according to the embodiment, one or more user terminals 7, and one or more administrator terminals 8.

The response profile evaluation device 1 is connected to the user terminal 7 and the administrator terminal 8 via a network. The response profile evaluation device 1 processes data received from the user terminal 7 and the administrator terminal 10 and sends the processed data to the user terminal 7 and the administrator terminal 8.

The user terminal 7 has a biological measurement sensor 11 measuring biological conditions of the user as a subject, a biological measurement device 12 controlling the biological measurement sensor 11, an input/output device 13, a communication device 14, and a notification device 15, and is used by the user.

The biological measurement sensor 11 has a heartbeat sensor 21 detecting a heartbeat interval (R-R interval, RRI) of the user, an electrodermal activity sensor 22 detecting an amount of perspiration of the user, and an acceleration sensor 23 detecting a motion of the user. As the heartbeat sensor 21, a sensor detecting heartbeats on the basis of, for example, electrocardiogram, photoplethysmography, pressure change, heart sound, or the like can be used.

The biological measurement sensor 11 is not limited to the above-described sensor, and a sensor detecting body temperature, eye blinks, eye movement, electromyogram, brain waves, or the like can be provided. As the biological measurement sensor 11, a wearable device the user can wear, a device provided on the inside of a smartphone the user can carry around, or the like can be used.

The biological measurement device 12 controls the biological measurement sensor 11 and generates biological measurement data from the biological conditions measured by the biological measurement sensor 11. The biological measurement data is data of the biological conditions of the user measured by the biological measurement sensor 11 and is biological information of the user. The biological measurement device 12 performs computing process and compressing process on the biological conditions as necessary to generate biological measurement data.

The input/output device 13 is a device having input devices such as a mouse, a keyboard, a touch panel, and a microphone and output devices such as a display and a speaker, and inputs/outputs data from/to the user terminal 7. The input device receives an input from the user and, for example, receives rating value data 81 measured by using a measurement scale to evaluate the subjective health condition of the user. The output device performs screen display on the display, for example, displays data obtained by the user terminal 7 and displays a notification to the user. The user terminal 7 performs screen display on the display.

Hereinafter, as a measurement scale used to measure the rating value data 81, a measurement scale of a continuous response type such as a visual analogue scale (VAS) is mainly assumed. In a measurement scale of the continuous response type, the rating value data 81 is measured as an arbitrary number from, for example, 0 to 100. In the embodiment, an example of electronically measuring a VAS by using a slider or the like displayed on the display of the user terminal 7 is employed. The VAS measurement in the embodiment can be also performed as follows. In a manner similar to a VAS measurement using a paper medium which is conventionally used in clinical practice, for example, the user makes a mark on a line segment drawn on a white paper sheet, and the value of the distance from one end of the line segment to the marked position is input by the input/output device 13.

The embodiment can be also applied to the rating value data 81 measured by the Likert scale as an ordinal scale of a multivalued discrete response type or the like. Particularly, when the number of options in an ordinal scale increases, it is known that the features of the ordinal scale become similar to those of a measurement scale of the continuous response type, and the embodiment can be easily applied.

The subjective health condition of the user is a condition of the body and mind of the user he/she is feeling and is a condition other than objective conditions obtained by measurement of sensors such as blood pressure and pulse. Examples of the subjective health condition are mind and body conditions obtained by measuring the degree of fatigue, stress, and a pain at a certain time point or in a certain period. For example, in the case of a pain, the subjective health condition can be measured by using a unipolar VAS as a VAS stating that the left end (or the lower end, the rating value is 0) is a no pain state and the right end (or the upper end, the rating value is 100) is the imaginable highest pain ever. An example of the subjective health condition is a body and health condition obtained by measuring natural emotion by using a slider (affective slider) for measuring a feeling dimension configured by arousal and valence. For example, in the case of measuring the valence by an affective slider, a bipolar VAS as a VAS stating that the left end (or the lower end, the rating value is 0) is a face mark in which negative feeling is reflected, the center (the rating value is 50) is a face mark in which neutral feeling is reflected, and the right end (or the upper end, the rating value is 100) is a face mark in which positive feeling is reflected can be used.

The communication device 14 executes process so that the user terminal 7 performs communication with the response profile evaluation device 1 and the administrator terminal 8 via the network 9.

The notification device 15 sends a notification to the user in accordance with a process of outputting a notification from the response profile evaluation device 1. For example, the notification device 15 displays a notification on the display of the input/output device 13. The notification device 15 may output a notification to the user by using, for example, vibration or sound. The notification device 15 may send a notification to the user also in the case where some change in the biological conditions of the user is detected by the biological measurement device 12.

Although the user terminal 7 is constructed as a single device in the embodiment, the user terminal 7 may not always be constructed as a single device. For example, when the input/output device 13 and the communication device 14 are provided for a smartphone, and the biological measurement device 12, the biological measurement sensor 11, and the notification device 15 are provided for a smartwatch, it can be regarded that one user terminal 7 is constructed by the smartphone and the smartwatch.

The administrator terminal 8 has an input/output device 31, a communication device 32, and a notification device 33 and is used by the administrator.

The input/output device 31 is a device having input devices such as a mouse, a keyboard, a touch panel, and a microphone and output devices such as a display and a speaker, and inputs/outputs data from/to the administrator terminal 8. The input device receives an input from the administrator. The output device performs screen display on the display, for example, displays data obtained by the administrator terminal 8 and displays a notification to the administrator. The administrator terminal 8 performs screen display on the display.

The communication device 32 executes process so that the administrator terminal 8 performs communication with the response profile evaluation device 1 and the user terminal 7 via the network 9.

The notification device 33 sends a notification to the administrator in accordance with process of outputting a notification from the response profile evaluation device 1. For example, the notification device 33 displays a notification on the display of the input/output device 31. The notification device 33 may output a notification to the administrator by using, for example, vibration or sound. The notification device 33 may send a notification to the user also in the case where some change in the biological conditions of the user is detected by the biological measurement device 12.

The response profile evaluation device 1 is constructed by a computer and has a processor 2, a memory 3, a storage device 4, an input/output device 5, and a communication device 6. The response profile evaluation device 1 has, as function units, a receiving unit 51, a preprocessing unit 52, an RP distribution estimating unit 53, an RP distribution evaluating unit 54, a result display unit 55, a data sampling unit 56, and a unit 57 for evaluating an RP distribution with a confidence interval. The details of the function units will be described later. The data sampling unit 56 and the unit 57 for evaluating an RP distribution with a confidence interval will be described in a second embodiment.

In the memory 3, a program for realizing the function units of the response profile evaluation device 1 is loaded. The program for realizing the function units is executed by the processor 2.

By executing the process in accordance with the program for realizing the function units, the processor 2 operates as a functioning unit providing a predetermined function. For example, by executing an RP distribution estimating program, the processor 2 functions as the RP distribution estimating unit 53. The other function units of the response profile evaluation device 1 are same as this example. Moreover, the processor 2 operates also as a functioning unit providing each of a plurality of processes executed by programs.

The storage device 4 stores data used by the above-described functioning units. For example, the storage device 4 stores the rating value data 81, subset rating value data 82, candidate RP distribution data 83, RP distribution data 84, balanced rating value data 85, RP distribution data 86 with a confidence interval, user characteristic data 87, RP distribution information 90, and distribution selection criterion information 91. The data will be described later. The balanced rating value data 85 and the RP distribution data 86 with a confidence interval will be described in the second embodiment.

The input/output device 5 has input devices and output devices. Example of the input devices include a mouse, a keyboard, a touch panel, and a microphone. Examples of the output devices include a display and a speaker.

The communication device 6 executes a process to make the response profile evaluation device 1 communicate with the user terminal 7 and the administrator terminal 8 via the network 9.

In the embodiment, an example that the user always wears or carries the user terminal 7 in daily life, and the biological measurement sensor 11 always operates will be described. The response profile evaluation device 1 in the embodiment can be used without being limited to the mode of the example. For example, the user may operate the biological measurement sensor 11 by using the user terminal 7 a few times a day at awakening time, bedtime, and the like.

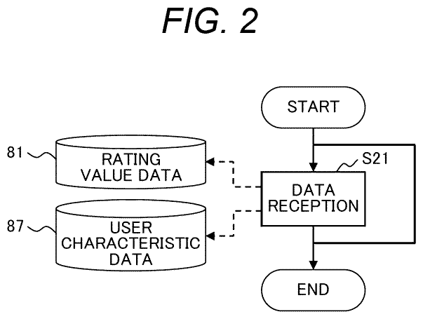

FIG. 2 is a flowchart illustrating an example of a process when the response profile evaluation device 1 receives data from the user terminal 7. This process is executed by the receiving unit 51 of the response profile evaluation device 1.

In data receiving process S21, when connection to the user terminal 7 is established via the network 9, the receiving unit 51 receives data from the user terminal 7. When the data receiving process S21 is started, the receiving unit 51 receives the rating value data 81 from the input/output device 13 of the user terminal 7 and stores it. The data receiving process S21 is continued until the connection between the receiving unit 51 and the user terminal 7 is interrupted.

In the case where the user characteristic data 87 is input to the user terminal 7 via the input/output device 13, in the data receiving process S21, the receiving unit 51 receives the user characteristic data 87 as well. The user characteristic data 87 includes the age, gender, user ID, and the like of the user.

Hereinafter, an example of measuring, as the rating value data 81, arousal and valence measured by a bipolar VAS at predetermined time in each day, and the degree of fatigue, stress, anxiety, depression, and sleeplessness measured by a unipolar VAS will be considered.

In the embodiment, an example that the response profile evaluation device 1 continuously performs the data receiving process S21 until the connection between the receiving unit 51 and the user terminal 7 is interrupted will be described. The response profile evaluation device 1 does not always have to perform the data receiving process S21 continuously. For example, only in the case where the user terminal 7 performs a process of transmitting data to the response profile evaluation device 1, the receiving unit 51 may establish connection to the user terminal 7 and perform the data receiving process S21. In this case, the user terminal 7 monitors the activity state of the user by the biological measurement device 12 and, when a predetermined event occurs, may send a notification to the user by using the notification device 15 to prompt the user to enter the rating value data 81 (a reply to an instruction).

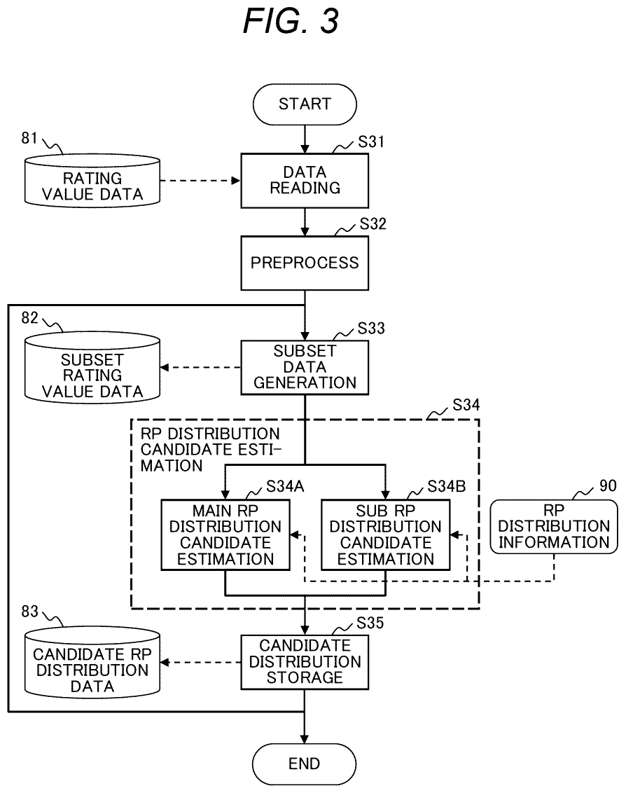

FIG. 3 is a flowchart illustrating an example of a process of estimating a response profile distribution (RP distribution) executed by the response profile evaluation device 1.

The response profile distribution is a statistical distribution indicating a response profile (RP). The response profile distribution is expressed by a distribution which is one statistical distribution or a mixture distribution of a plurality of statistical distributions, and expressed by a parameter group characterizing the distribution (a group of features determining the shape of the distribution).

In a data reading process S31, the receiving unit 51 of the response profile evaluation device 1 receives the rating value data 81 of the user.

In a preprocess S32, the preprocessing unit 52 of the response profile evaluation device 1 performs a preprocess of the rating value data 81 which is input. In the case where a subsequent process is performed for each user, first, the preprocessing unit 52 extracts the user characteristic data 87 for only the user as a process target from the user characteristic data 87 (FIG. 2) received by the receiving unit 52.

The preprocessing unit 52 performs a preprocess according to the rating value data 81 which is input. For example, in the case where a VAS is recorded that a domain is from 0 to 100, a normalization process is performed on the rating value data 81 so that the domain of the VAS lies in the range from 0 to 1. For example, in the case where a plurality of VASs are recorded by types and the domains are different among the VASs, the normalization process is performed on the rating value data 81 so that the domains lie in the same range. In a VAS or the like, an effort minimization tendency that a respondent avoids an effort for an answering action and answers non-seriously without reading an item sentence may occur. The preprocessing unit 52 may perform a noise eliminating process of detecting a response in which the effort minimization tendency is visible from response required time included in the rating value data 81 and the like and eliminating the response from a process target.

In a subsequent RP distribution candidate estimating process S34, there is a case that the domain of the rating value data 81 has to be larger than 0 and less than 1. In this case, for example, the preprocessing unit 52 may scale extracted rating value data 81 (d) of the user to rating value data 81 (d′) as a process of converting the domain from the range [0, 1] of 0 or larger and 1 or smaller to the range (0, 1) of larger than 0 and less than 1. When it is assumed that the total number of data samples of the rating value data 81 (d) is n, the scaling is expressed by a formula d′={d (n−1)+0.5}/n. At the time of referring to a domain, brackets [ ] indicate a range including values in the brackets (closed interval), and parentheses ( ) indicate a range which does not include values in the parentheses (open interval).

In a subset data generating process S33, the preprocessing unit 52 divides the preprocessed rating value data 81 by data ranges to generate the subset rating value data 82 for estimating a main RP distribution candidate and a sub RP distribution candidate. The main RP distribution candidate and the sub RP distribution candidate will be described later.

First, division of the rating value data 81 will be described.

From the study of a response style (response tendency) in the Likert scale as a measurement scale of a multivalued discrete response type, various typical response styles are known. Well-known response styles are an acquiescence response style (ARS) biased to the highest rating regardless of an item or an instruction, a dis-acquiescence response style (DRS) biased to the lowest rating, and an extreme response style (ERS) biased to both extreme ratings. Among Japanese, a mid-point response style (MRS) biased to the neighborhood of the center, or the like is often recognized.

Those response tendencies are recognized in similar manners also in the case of using a measurement scale of a continuous response type such as a VAS. Further, in MRSs, there is a case that a bimodal response style (bimodal MRS, BiMRS) having two distribution modes (peaks) around the center is observed. The BiMRS is often observed in the case where a bipolar VAS in which the center of a measurement scale is set as the neutral and contradictory concepts are set at both poles with respect to the center is used, and in the case of using the semantic difference method of giving an adjective instruction indicating opposite concepts at both poles as an instruction even in a Likert scale and performing measurement. Hereinafter, as an example of a response profile (RP) generated in the rating value data 81, an example of assuming an ARS, a DRS, an ERS, an MRS, and a BiMRS will be described.

Among RPs, in an ARS, a DRS, an ERS, an MRS, and a BiMRS, ranges in which the rating value data 81 locally exists and is easily observed are different. Therefore, in the case where an RP in which the rating value data 81 locally exists in a plurality of positions is assumed, in the subset data generating process S33, the preprocessing unit 52 may divide the rating value data 81 by a data range of the domain to generate the subset rating value data 82.

For example, in the case of the rating value data 81 whose domain is normalized to a range which is larger than 0 and less than 1 (0, 1), it is considered that, in an MRS and a BiMRS, the rating value data 81 is expressed in neighborhood of the center of the domain (that is, neighborhood of 0.5) and, in an ARS, a DRS, and an ERS, the rating value data 81 is expressed in neighborhoods of the upper limit and the lower limit of the domain (that is, neighborhoods of 0 and 1). Consequently, the preprocessing unit 52 can set the neighborhood as a range determined by a preliminarily set threshold th, and divide the rating value data 81 into main subset rating value data as data in the neighborhood of the center of the domain and sub subset rating value data as data in the neighborhood of the upper limit and the neighborhood of the lower limit of the domain. Specifically, using the preliminarily determined threshold th, the preprocessing unit 52 can divide the rating value data 81 into subset rating value data 82 made by main subset rating value data whose domain is the range [th, 1-th] and sub subset rating value data whose domains are the range (0, th) and the range (1-th, 1). For each of the subset rating value data 82, a subsequent RP distribution candidate estimating process S34 which will be described later may be executed.

Although the neighborhood is determined by one threshold th which is preliminarily determined in the above, the method of determining a threshold is not limited to this example. For example, ranges as neighborhoods of the upper limit and the lower limit of the domain may be determined by using a threshold th1 related to the lower limit and a threshold th2 related to the upper limit. Specifically, in this case, using the preliminarily determined threshold th1 and th2, the preprocessing unit 52 may divide the rating value data 81 into the subset rating value data 82 made by main subset rating value data whose domain is the range [th1, th2] and sub subset rating value data whose domains are the range (0, th1) and the range (th2, 1).

Hereinafter, an example that the rating value data 81 is divided to the subset rating value data 82, and the subset rating value data 82 is made by main subset rating value data and sub subset rating value data determined by the domain using the preliminarily determined threshold th will be described.

The threshold th can be determined to an arbitrary value. For example, in the rating value data 81 whose domain is normalized to the range [0, 1] or the range (0, 1), the threshold th can be set to a value chosen from the range from 0.05 to 0.30 or the like by reference to an experiment result which will be described later.

In the RP distribution candidate estimating process S34, the RP distribution estimating unit 53 estimates a plurality of response profile distribution candidates (RP distribution candidates) on the basis of each of the pieces of the subset rating value data 82. The RP distribution candidate is a candidate of a statistical distribution expressing the RP of the user, and can be estimated by fitting to a statistical distribution expressed in a candidate RP distribution template.

In the RP distribution candidate estimating process S34, the RP distribution estimating unit 53 approximates the distribution shape of the subset rating value data 82 by a statistical distribution on the basis of a candidate RP distribution template stored in the RP distribution information 90. The candidate RP distribution template includes the kind of a statistical distribution (for example, a normal distribution and a beta distribution) expressing distributions which can express an RP (for example, an ERS and a BiMRS) and the range of values of parameters θ determining the shape of the distribution. The parameter θ is a feature determining the shape of the distribution and, for example, when the kind of the statistical distribution is the normal distribution, is an average, standard deviation, or the like.

Hereinafter, an example of estimating an RP distribution candidate as a candidate of a statistical distribution characterizing an RP of the user through fitting to a parametric statistical distribution in which the shape of the distribution is characterized by the parameter θ by the RP distribution estimating unit 53 will be described. In this fitting, a histogram of the subset rating value data 82 is fit to a probability density function of the statistical distribution. By fitting a cumulative histogram of the subset rating value data 82 to a cumulative probability density function of the statistical distribution, an RP distribution candidate may be estimated. In the case where an ordinal scale of a multivalued discrete response type such as the Likert scale is used as the rating value data 81, in place of fitting to the probability density function of the statistical distribution, by fitting to a probability mass function or a cumulative probability mass function of an appropriate discrete statistical distribution, an RP distribution candidate may be estimated.

Hereinafter, the RP distribution candidate estimating process S34 will be described by dividing into a main RP distribution candidate estimating process S34A and a sub RP distribution candidate estimating process S34B. The RP distribution estimating unit 53 estimates a plurality of RP distribution candidates including at least one of a main RP distribution candidate expressing an RP appearing in the neighborhood of the center of the rating value data 81 and a sub RP distribution candidate expressing an RP appearing in at least one of the neighborhood of the upper limit and the neighborhood of the lower limit of the rating value data 81.

First, the main RP distribution candidate estimating process S34A will be described. In the main RP distribution candidate estimating process S34A, the RP distribution estimating unit 53 estimates a response profile distribution candidate (main RP distribution candidate Main) for the main subset rating value data. The parameter θ determining the shape of the distribution of a main RP distribution candidate Main is set as a main RP parameter θ_{Main}.

The RP distribution estimating unit 53 estimates the main RP parameter θ_{Main} of the main RP distribution candidate Main. It is assumed that, in the RP distribution information 90, as candidate RP distribution templates, a candidate RP distribution template of an MRS and a candidate RP distribution template of a BiMRS are stored. It is also assumed that a candidate RP distribution template of a uniform distribution having no peaks is stored as a candidate RP distribution template in the RP distribution information 90. An MRS can be expressed by a unimodal continuous distribution. A BiMRS can be expressed by a mixture distribution made by two continuous distributions (a distribution having two peaks).

The RP distribution estimating unit 53 estimates the main RP distribution candidate Main by using, for example, a normal distribution Normal (μ, σ) in which a main RP parameter θ_{Main} is an average u and standard deviation σ, a beta distribution Beta (α, β) in which the main RP parameter θ_{Main} is parameters α and β, or a mixture distribution obtained by two distributions as a candidate RP distribution template, by fitting to the candidate RP distribution template. For example, in the case of fitting to a mixture distribution of two beta distributions L and R, the main RP distribution candidate Main is expressed as BetaMixture (w_{L}, α_{L}, β_{L}, w_{R}=1−w_{L}, α_{R}, β_{R}). w denotes the mixture ratio of the two beta distributions, and indexes L and R express parameters of the respective beta distributions. For parameter fitting, a known method can be used. For example, in the case of an MRS, the maximum-likelihood method or Bayes' estimation can be used. In the case of a BiMRS, the expectation-maximization algorithm or a similar algorithm can be used.

In such a manner, the RP distribution estimating unit 53 estimates the main RP parameter θ_{Main} characterizing the main RP distribution candidate Main of each of the main subset rating value data and, by using it, estimates the main RP distribution candidate Main.

The sub RP distribution candidate estimating process S34B will now be described. In the sub RP distribution candidate estimating process S34B, the RP distribution estimating unit 53 estimates a response profile distribution candidate (sub RP distribution candidate DistSub) for the sub subset rating value data. A parameter θ determining the shape of the distribution of the sub RP distribution candidate DistSub is set as a sub RP parameter θ_{DistSub}.

The RP distribution estimating unit 53 estimates a sub RP parameter θ_{DistSub} of the sub RP distribution candidate DistSub. Since the ARS, the DRS, and the ERS do not have peaks, for example, the RP distribution estimating unit 53 sets a beta distribution Beta (α_{ADE}, β_{ADE}) which is a continuous distribution that can be flexibly expressed and is expressed by using parameters α_{ADE} and β_{ADE} as a candidate RP distribution template. The RP distribution estimating unit 53 estimates the sub RP distribution candidate DistSub by fitting to the candidate RP distribution template. The RP distribution estimating unit 53 estimates a sub RP parameter θ_{DistSub} characterizing each of sub RP distribution candidates DistSub by parameter fitting in which α_{ADE} and β_{ADE} are restricted to secure the distribution shape of the ARS, the DRS, and the ERS with respect to the sub subset rating value data and, by using it, estimates the sub RP distribution candidate DistSub.

In a candidate distribution storing process S35, the RP distribution estimating unit 53 stores the candidate RP distribution data 83. The candidate RP distribution data 83 includes the main RP distribution candidate Main and the sub RP distribution candidate DistSub obtained by the RP distribution candidate estimating process S34 (the main RP distribution candidate estimating process S34A and the sub RP distribution candidate estimating process S34B), and the main RP parameter θ_{Main} and the sub RP parameter θ_{DistSub} characterizing them. In the case where a uniform distribution having no peaks is obtained as the main RP distribution candidate Main, a feature characterizing the uniform distribution is stored as the candidate RP distribution data 83.

The data localization range of the main RP distribution candidate Main and that of the sub RP distribution candidate DisSub are hardly overlap each other. Consequently, by dividing the rating value data 81 to the subset rating value data 82 made by the main subset rating value data and the sub subset rating value data and obtaining the candidate RP distribution data 83 by using the subset rating value data 82, the precision of estimating the RP distribution candidate by the RP distribution estimating unit 53 can be improved.

In the above, the example of extracting only specific user characteristic data 87 and estimating an RP distribution candidate using the histogram of the rating value data of only a specific user as a target has been described. The process of estimating an RP distribution candidate is not limited to the process on a specific user. For example, in the case where a plurality of users respond to question items of a plurality of kinds and the number of responses are almost equal among the plurality of users, a plurality of item response theory models each having an RP as a latent variable are provided as candidate RP distribution templates and an RP distribution candidate may be estimated. In this case, when the measurement scale of the continuous response type such as a VAS is rating value data, an RP distribution candidate and its RP parameter θ may be estimated by using a model extended by introducing a parameter indicating an RP to a beta response model.

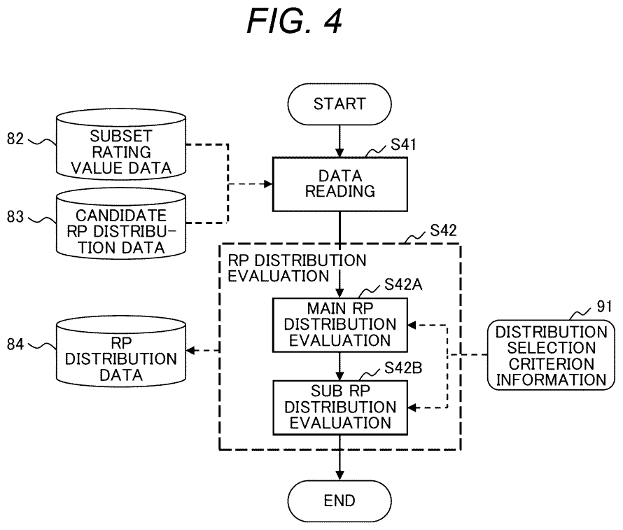

FIG. 4 is a flowchart illustrating an example of an RP distribution evaluating process performed by the response profile evaluation device 1. In the RP distribution evaluating process, based on the rating value data 81 or the subset rating value data 82 and a plurality of RP distribution candidates, an RP distribution adapted to characterize the RP of the user (that is, an RP distribution adapted to expression of the RP of the user) is obtained by using an evaluation index. Hereinafter, an example of obtaining an RP distribution adapted to the expression of the RP of the user by using the subset rating value data 82 will be described.

In a data reading process S41, the receiving unit 51 of the response profile evaluation device 1 receives the subset rating value data 82 and the candidate RP distribution data 83.

In an RP distribution evaluating process S42, the RP distribution evaluating unit 54 selects an RP distribution adapted to express the RP of the user from a plurality of RP distribution candidates by using the subset rating value data 82. The RP distribution evaluating unit 54 can select, for example, one RP distribution, a plurality of RP distributions, or a combination of a plurality of RP distributions as an RP distribution adapted to express the RP of the user.

Concretely, the RP distribution evaluating unit 54 selects an evaluation index for determining the RP distribution adapted to characterize the RP of the user on the basis of the combination of the candidate RP distribution data 83. The evaluation index is an index for evaluating the degree of fitting of the distribution of the rating value data 81 (subset rating value data 82) to an RP distribution candidate and is selected so that the evaluation index satisfies a condition determined by a predetermined threshold. The RP distribution evaluating unit 54 sets a distribution expressed by an RP distribution candidate (a main RP distribution candidate and a sub RP distribution candidate) corresponding to the selected evaluation index as an RP distribution adapted to characterize the RP of the user. In the embodiment, it is assumed that the RP distribution of the user is expressed by a mixture distribution in which a statistical distribution adapted to characterize the RP of the user is mixed.

In the embodiment, an example of assuming the case where a main RP distribution candidate and a sub RP distribution candidate are estimated and performing the RP distribution evaluating process S42 being separated to a main RP distribution evaluating process S42A as a process for a main RP distribution candidate and a sub RP distribution evaluating process S42B as a process for a sub RP distribution candidate will be described. In the embodiment, particularly, a combination of a main RP distribution candidate and a sub RP distribution candidate is not exhaustively searched but an example of a hierarchical process of selecting a suitable RP distribution from main RP distribution candidates Main as a main RP distribution Main and, after that, evaluating a combination with a sub RP distribution candidate only with respect to the selected main RP distribution Main will be described. It is also possible to select RP distributions respectively adapted to a main RP distribution candidate and a sub RP distribution candidate in parallel and evaluate only a combination of the main RP distribution and the sub RP distribution obtained. By executing the hierarchical process or the parallel process in place of the exhaustive search process, the evaluation space of the candidate RP distribution evaluation can be narrowed, and an effect that the calculation amount and the process time are reduced is obtained.

The RP distribution evaluating unit 54 performs the main RP distribution evaluating process S42A first as the RP distribution evaluating process S42. In the case where the MRS, the BiMRS, or a uniform distribution having no peak is assumed as a main RP, in the main RP distribution evaluating process S42A, an adapted kind of a statistical distribution is determined on the basis of the evaluation index given to the distribution selection criterion information 91. In the distribution selection criterion information 91, an item of an evaluation index is given in advance.

The evaluation index may be selected by comparing a main RP distribution candidate characterized by the main RP parameters @Main and a histogram of the main subset rating value data from a necessary viewpoint. For example, as an evaluation index, to evaluate fitting of the histogram of the main subset rating value data to the main RP distribution candidate, a likelihood related statistic amount or an information criterion may be employed. As the likelihood related statistic amount, for example, likelihood, logarithmic likelihood, likelihood ratio, degree of deviation, or the like can be used. As the information criterion, for example, Akaike's information criterion (AIC), Bayesian information criterion (BIC), widely applicable information criterion (WAIC), widely applicable BIC (WBIC), or the like can be used. As an evaluation index, to evaluate an obtained distribution shape, Kullback-Leibler divergence which measures distributional similarity may be employed.

In the case where the MRS and the BiMRS are assumed as a main RP and the BiMRS is expressed by a mixture distribution, when the main RP distribution is evaluated simply by the information criterion, even if the degree of separation of peaks (the degree of separation of two modes) in the mixture distribution is not so high, the BiMRS as a complicated RP distribution tends to be selected. It is desirable that a BiMRS having a large number of parameters is selected only in the case where the degree of separation of two modes is high. Consequently, it is also possible to set the degree of separation of two modes as an evaluation index so that the BiMRS is selected only in the case where the degree of separation of two modes of the BiMRS estimated as the main RP distribution is remarkably high, and to evaluate the main RP distribution on the basis of whether or not the evaluation index exceeds the minimum allowable threshold accept_bidist. For example, in the case of approximating the BiMRS by a mixture beta distribution in which the number of mixture is two, the degree of separation of two modes can be calculated by the difference between the mode values of two fractional distributions constructing the mixture distribution of the BiMRS.

By the above, for example, only in the case where the degree of separation of peaks (the degree of separation of two modes) of the mixture distribution as the evaluation index is larger than the minimum allowable threshold accept_bidist and the AIC is small, the BiMRS is selected as the main RP distribution. In the other case, the main RP distribution Main and the main RP parameter θ_{Main} characterizing the main RP distribution are selected as an MRS or a uniform distribution having no peaks (that is, the main RP distribution does not exist).

After executing the main RP distribution evaluating process S42A, the RP distribution evaluating unit 54 executes the sub RP distribution evaluating process S42B. In the sub RP distribution evaluating process S42B, an optimum mixture ratio w_{ADE} of the main RP distribution Main characterized by the main RP parameter θ_{Main} and the sub RP distribution DistSub characterized by each of the sub RP parameters θ_{DistSub} is estimated, and the evaluation index when the mixture ratio of the distributions is w_{ADE} is calculated. The optimum mixture ratio w_{ADE} of the distributions can be estimated by using the same method as the method of fitting to the statistical distribution described in the RP distribution candidate estimating process S34. Each evaluation index obtained and each evaluation index in the case of selecting the main RP distribution as an RP distribution are compared, and a condition of an excellent evaluation index is employed.

For example, in the case of using an information criterion as the evaluation index, when a mixture distribution of a main RP distribution and a sub RP distribution is employed, if the information criterion improves, the mixture distribution is selected as an RP distribution expressing the RP of the user. If it does not improve, the main RP distribution is selected as an RP distribution expressing the RP of the user. When the information criterion improves the most in the case of selecting the BiMRS as the main RP distribution Main and selecting the ARS as the sub RP distribution DisSub for a fractional distribution of the mixture distribution, an RP distribution ResponseProfile (θ) is expressed as ResponseProfile (θ)=w_{ADE}×DistSub (θ_{DistSub})+ (1−w_{ADE})×Main (θ_{Main}), and an RP distribution parameter θᵤₛₑᵣ is expressed as θᵤₛₑᵣ={θ_{DistSub}, θ_{Main}}={w_{ADE}, α_{ARS}, β_{ARS}, w_{L}, β_{L}, β_{L}, w_{R}, α_{R}, β_{R}}. The RP distribution estimating unit 54 stores the obtained RP distribution parameter θᵤₛₑᵣ, and the information of the statistical distribution kind as the RP distribution data 84.

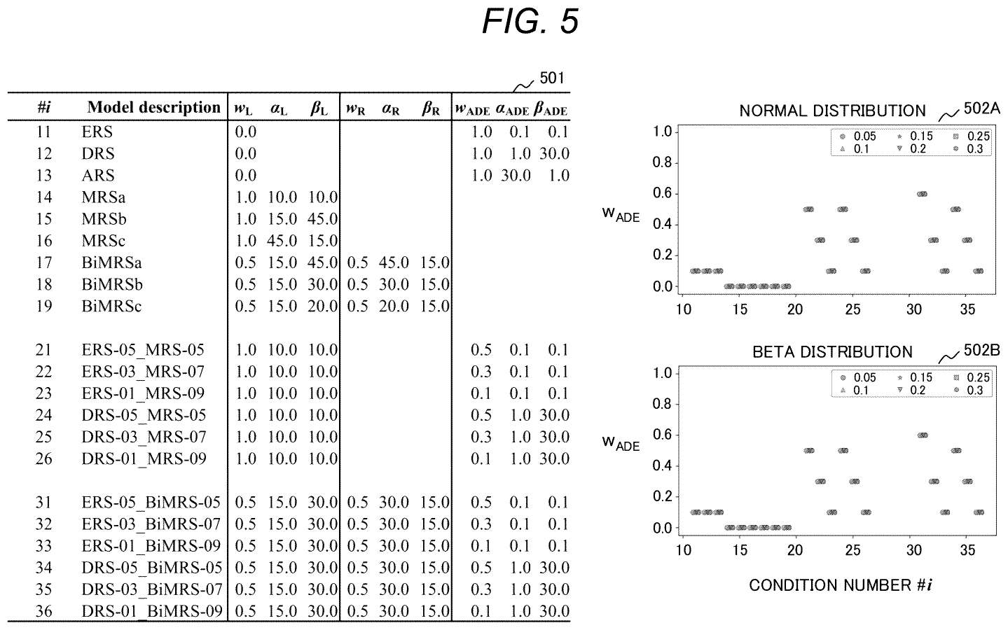

FIG. 5 is a result of an experiment of the RP distribution estimation (FIG. 3) and the RP distribution evaluation (FIG. 4) performed by using the response profile evaluation device 1 of the embodiment. The experiment is performed by using artificial data of the rating value data 81.

In this experiment, in the case where the RP distribution is expressed as ResponseProfile (θ)=w_{ADE}×DistSub(θ_{DistSub})+(1−w_{ADE})×Main (θ_{Main}), and an RP distribution parameter is expressed as θᵤₛₑᵣ={θ_{DistSub}, θ_{Main}}={w_{ADE}, α_{ARS}, β_{ARS}, w_{L}, α_{L}, β_{L}, w_{R}, α_{R}, β_{R}}, particularly, the influence of the threshold th used to generate the subset rating value data 82 by using artificial data of the rating value data 81 generated from a statistical distribution whose true parameter is known was evaluated.

A table 501 of FIG. 5 is a table expressing an example of artificial data used for an experiment and illustrates an RP distribution and its RP distribution parameter with respect to each of conditions of numbers #11 to #36. Data of only 1000 samples is extracted from each beta mixture distribution defined by the RP distribution parameter illustrated in the table 501, and the AIC and the degree of separation of peaks (the degree of separation of modes) of the mixture distribution are employed as an evaluation index. By evaluating the RP distribution parameter θᵤₛₑᵣ in the case where the minimum allowable threshold accept_bidist of the degree of separation of two modes is set to 0.0 and a continuous distribution which approximates a main RP distribution is set to a normal distribution or a beta distribution, robustness of an estimate value for the threshold th and the kind of the continuous distribution was evaluated.

Graphs 502A and 502B illustrate results of the mixture ratio w_{ADE} of the main RP distribution Main and the sub RP distribution DistSub in the RP distribution parameter θᵤₛₑᵣ as an experiment result. In the experiment, the continuous distribution (the kind of the statistical distribution selected as a candidate RP distribution template) approximating the main RP distribution Main is a normal distribution and a beta distribution. The graph 502A illustrates the case where the kind of the distribution is a normal distribution, and the graph 502B illustrates the case where the kind of the distribution is a beta distribution. In the experiment, the value of the threshold th was changed as 0.05, 0.15, 0.25, 0.1, 0.2, and 0.3.

It can be recognized from the graphs 502A and 502B that when the RP distribution parameter θᵤₛₑᵣ is evaluated by the method according to the embodiment, although diremption from a true value occurs under a part of conditions, regardless that the kind of the continuous distribution is a normal distribution or a beta distribution, when the threshold th lies at least in the range from 0.05 to 0.3, there is no large change in the estimation result of w_{ADE} and the estimation result is robust. Including a part of conditions where diremption from a true value occurs, the framework of a selected RP distribution reproduces the distribution of artificial data. That is, in the embodiment, it is insusceptible to a change in the RP distribution parameter θᵤₛₑᵣ, the almost same RP can be obtained as the response profile (RP) of the user, and a stable result can be obtained.

As described above, according to the embodiment, even the rating value data 81 measured by using a measurement scale of a continuous response type such as a VAS, a response profile (RP) generated in the rating value data 81 can be quantitatively evaluated.

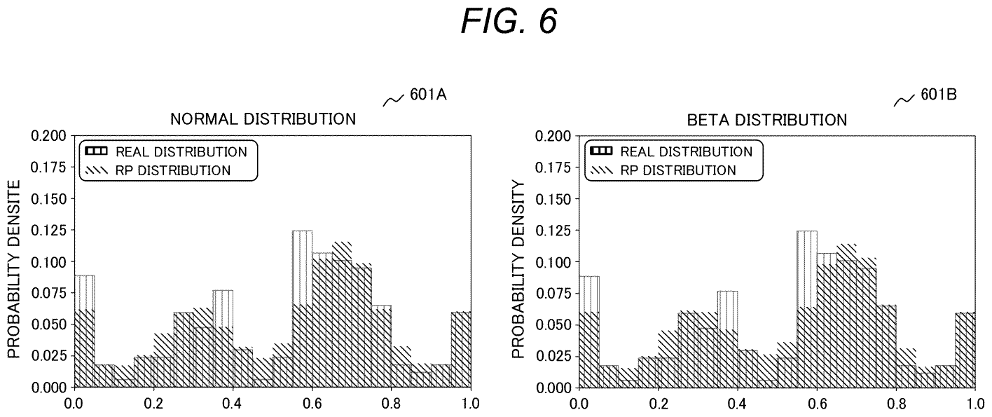

FIG. 6 is a diagram illustrating an example of the RP distribution data 84 of the user, which is displayed on the user terminal 7 or the administrator terminal 8. Graphs 601A and 601B illustrated in FIG. 6 are an example of display of an RP distribution obtained by applying the method according to the embodiment.

Graphs 601A and 601B are an example of displaying a histogram (real distribution) of the rating value data 81 and a probability density function of an RP distribution as a histogram having a bin width 0.05. The graph 601A illustrates an RP distribution obtained by using a continuous distribution approximating a main RP distribution as a normal distribution. The graph 601B illustrates an RP distribution obtained by using a continuous distribution approximating a main RP distribution as a normal distribution. The graph 601B illustrates an RP distribution obtained using a continuous distribution approximating a main RP distribution as a beta distribution. In the graphs 601A and 601B, vertically hatched histograms are histograms expressing actual rating value data made by a bipolar VAS and a unipolar VAS of a plurality of kinds for a certain user. Obliquely hatched histograms are histograms displaying a probability density function of an RP distribution estimated by the method (an RP distribution obtained on assumption that W_{ADE} is 0.3).

In the example illustrated in FIG. 6, concentration of rating values is seen at both ends of the domain and concentration of rating values of both modes is seen in positions sandwiching the center part. Consequently, it can be interpreted as an example where the main RP distribution is the BiMRS, and the sub RP distribution is the ERS.

As a result of applying the method, in the case where the continuous distribution is set as a normal distribution and also in the case where it is set as a beta distribution, it is estimated that the RP can be expressed by the mixture distribution in which w_{ADE} is 0.3 and which is made by the ERS and the BiMRS, and it can be well recognized that the RP distribution approximating the rating value data 81 is obtained. By the method, effects are obtained such that the RP can be quantitively evaluated by using a small amount of RP distribution parameters θᵤₛₑᵣ, and an RP generated in the rating value data 81 measured by using a measurement scale of mainly a continuous response type such as a VAS can be quantitively evaluated and compared.

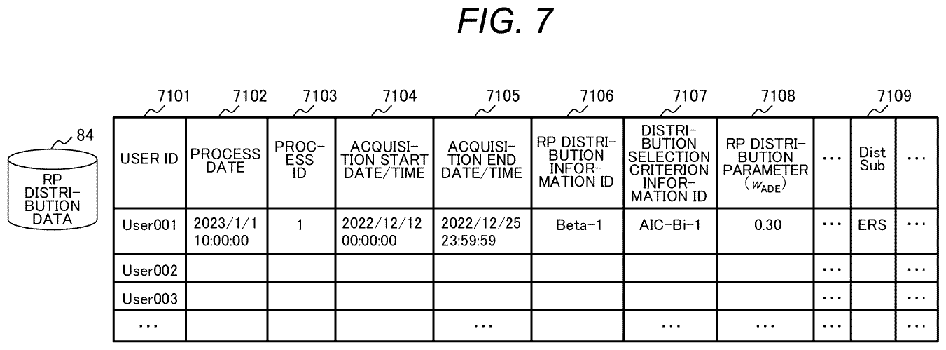

FIG. 7 is a diagram illustrating an example of a data structure of the RP distribution data 84. The RP distribution data 84 has, typically, user ID 7101, process date 7102, process ID 7103 for unconditionally specifying a process result, processed rating value data acquisition start date and time 7104, acquisition end date and time 7105, RP distribution information ID 7106 for specifying the RP distribution information 90 used for processing, distribution selection criterion information ID 7107 for specifying the distribution selection criterion information 91, and an RP distribution parameter 7108 characterizing an RP distribution such as w_{ADE}. The RP distribution data 84 may have, for example, a DistSub 7109 indicating the kind of a sub RP distribution as an index characterizing the RP distribution.

In the RP distribution data 84 in FIG. 7, a sub RP distribution is illustrated but a main RP distribution is not illustrated. The kind (for example, a distribution shape) of a main RP distribution can be grasped from the RP distribution information ID 7106 specifying the RP distribution information 90 used for processing.

The result display unit 55 can display a result calculated by the response profile evaluation device 1 on the display of the user terminal 7 or the administrator terminal 8. For example, the result display unit 55 can display at least one of an RP distribution (for example, FIG. 6) and a parameter group characterizing the RP distribution (for example, the RP distribution parameter 7108 in FIG. 7) on the display.

As described above, the response profile evaluation device 1 according to the embodiment has the receiving unit 51 receiving the rating value data 81 of the user, the RP distribution estimating unit 53 estimating a plurality of RP distribution candidates expressing a response profile (RP) as a response tendency of the user on the basis of the rating value data 81, and the RP distribution evaluating unit 54 selecting an RP distribution adapted to express the RP of the user from a plurality of RP distribution candidates on the basis of the rating value data 81 and a plurality of RP distribution candidates. The response profile evaluation device 1 according to the embodiment may have the result display unit 55 displaying at least one of an RP distribution and a parameter group characterizing the RP distribution.

With the configuration, the response profile evaluation device 1 according to the embodiment has an effect that an RP can be quantitively evaluated in a manner such that it can be compared with other users also for the rating value data 81 measured by using a measurement scale of a continuous response type such as a VAS which often includes items of types of different measurement scales such as a unipolar scale and a bipolar scale. Further, from this effect, an effect contributing to realization of intersubject analysis of a psychology measurement value in which the influence of an RP is considered is obtained secondarily.

### Second Embodiment

A response profile evaluation device, a response profile evaluation method, and a response profile evaluation program according to a second embodiment of the present invention will be described. Hereinafter, points different from the first embodiment, of the second embodiment will be described.

In the case of repetitively measuring VASs of a plurality of kinds with time daily, a tendency may occur in the number of responses and the response kinds in accordance with the kind of a VAS to be measured. A response profile (RP) or a response style is a tendency in the response tendency consistent to each subject occurring regardless of an instruction (that is, content independence or subject dependence). Consequently, in the configuration of the first embodiment, it is difficult to assume content independence in the case where there is tendency in the number of responses or the kinds of responses and there is the possibility that an RP influenced by a VAS of, for example, a large number of responses is estimated. In view of the problem, the second embodiment is characterized by further providing a process of artificially securing the content independence.

The configuration of the response profile evaluation device 1 according to the second embodiment is illustrated in FIG. 1.

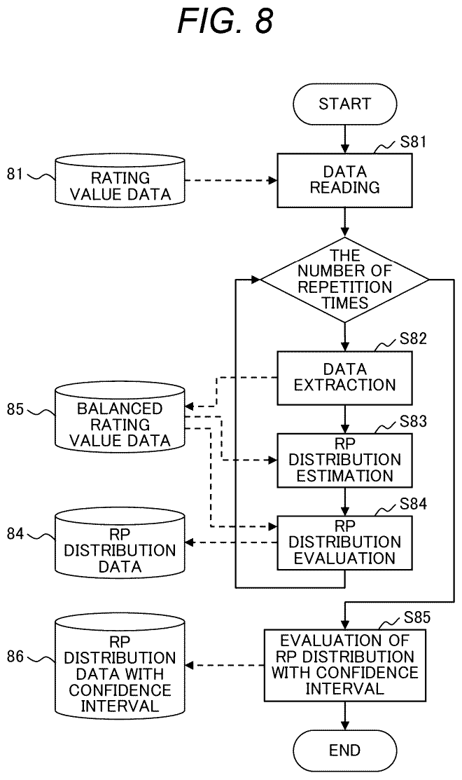

FIG. 8 is a flowchart illustrating an example of a series of processes of evaluating an RP distribution with a confidence interval performed by the response profile evaluation device 1 in the second embodiment.

In a data reading process S81, the receiving unit 51 of the response profile evaluation device 1 receives the rating value data 81.

After that, each processing unit executes a series of processes which will be described later only by a predetermined number of repetitive times B.

In a data extracting process S82, the data sampling unit 56 generates the balanced rating value data 85 as the rating value data 81 artificially securing the content independence. The data sampling unit 56 restores and extracts the rating value data 81 made of a plurality of different response items obtained from the user to resample the rating value data 81, thereby artificially eliminating the difference of the numbers of responses among the response items. The rating value data 81 made of a plurality of different response items includes, for example, the rating value data 81 in the case of different kinds such as a unipolar VAS and a bipolar VAS and the rating value data 81 in the case where the kinds of VASs are the same but instructions are different.

The data sampling unit 56 resamples the rating value data 81 to generate balanced rating value data 85 in which the kinds of the rating value data 81 or the number of cases of the rating value data 81 are artificially balanced. For example, in the case where the measurement scale is only a bipolar scale or a unipolar scale, sampling with replacement of the predetermined number of samples is performed. In the case where both a bipolar scale and a unipolar scale exist, the numbers of samples in each of the kinds of VASs are balanced by sampling with replacement in the first layer, and the difference of the numbers of samples between the bipolar scale and the unipolar scale is balanced by sampling with replacement in the second layer. In such a manner, the balanced rating value data 85 including the same number of responses in each response item can be generated. By the process, a data set including the same number of times and a plurality of response items is obtained, and a plurality of pieces of rating value data in which the influence of a tendency in response items and the numbers of times of responses is reduced, that is, rating value data whose content independence is artificially secured can be artificially generated.

Subsequently, using the balanced rating value data 85 as an input, an RP distribution estimating process S83 illustrated in FIG. 3 and an RP distribution evaluating process S84 illustrated in FIG. 4 are performed, thereby calculating the RP distribution data 84 for each piece of the balanced rating value data 85. The processes correspond to generation of bootstrap samples only by the predetermined number B of repetition times by the bootstrapping method and configuration of a bootstrap distribution of an RP distribution parameters θ_{user, i}.

After that, a process S85 of evaluating an RP distribution with a confidence interval is performed. The unit 57 for evaluating an RP distribution with a confidence interval performs a process S85 of evaluating an RP distribution with a confidence interval on the RP distribution data 84 in which the bootstrap distribution of the obtained RP distribution parameter θ_{user, i} is expressed. In the process S85 of evaluating an RP distribution with a confidence interval, the unit 57 for evaluating an RP distribution with a confidence interval calculates an RP distribution with a confidence interval by calculating a bootstrap statistic of a bootstrap distribution constructed by the number of repetition times (that is, B pieces of) RP distribution parameters θ_{user, i}. The RP distribution with a confidence interval is an RP distribution indicated with a plausible range (confidence interval) and is expressed by the RP distribution data 86 with a confidence interval.

For example, by calculating a mean of a bootstrap distribution and a 95% confidence interval by the normal distribution approximation method, the RP distribution parameter θ_{user, i} can be calculated with a confidence interval. Without being limited to the normal distribution approximation method, the RP distribution data 86 with a confidence interval may be calculated by a known method such as the percentile method. Consequently, in a form that the possibility of being affected by a rating value of the kind of the large number of times of responses is reduced and it can be also considered how much an estimation result may fluctuate, the RP of the user can be estimated.

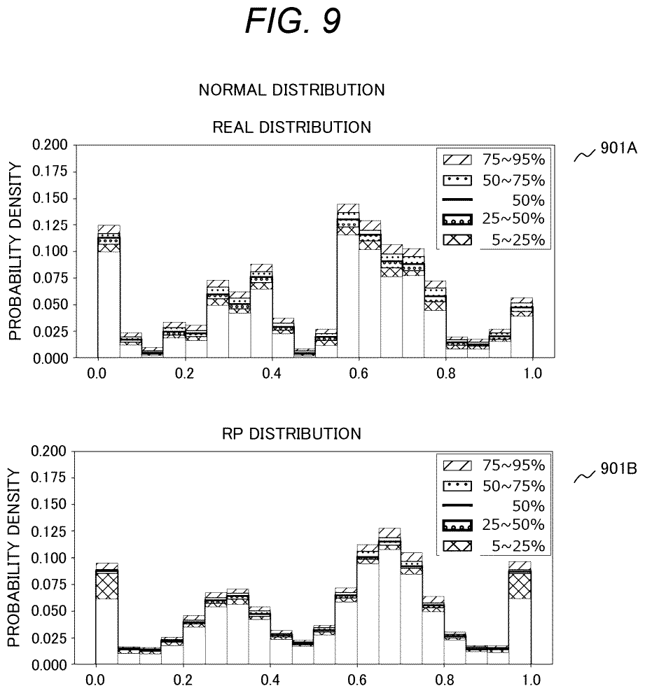

FIG. 9 is a diagram illustrating an example of the RP distribution data 86 with a confidence interval of the user, which is displayed on the user terminal 7 or the administrator terminal 8. The example of FIG. 9 illustrates a histogram 901A (real distribution) of each of pieces of the balanced rating value data 85 in the case where the number of repetition times is 1000, the type of the continuous distribution is a normal distribution, and the method described in the second embodiment is applied, and a probability density function 901B of an RP distribution corresponding to the histogram 901A as histograms having a bin width of 0.05.

In the histograms of FIG. 9, an envelope drawn by the PR distribution parameter θ_{user, i} of the median of the number of repetition times (that is, B pieces) of the PR distribution parameter θ_{user, i} is expressed by the thick line (the thick line indicating 50%) at the upper end of the region indicating 25 to 50%, and the total of a plurality of hatched regions is a range of an RP distribution in the 95% range (the plausible range of the RP distribution) of the PR distribution parameters θ_{user, i}.

By repeating the sampling with replacement, the occurrence frequency of the balanced rating value data 85 fluctuates as in the histogram 901A. This fluctuation is reflected, and an RP distribution shape estimated as an RP distribution with a confidence interval also fluctuates like the probability density function 901B. According to the method of the embodiment, even from the rating value data 81 including a tendency in the number of times of responses or the like, the RP of the user can be estimated with a confidence interval.

As described above, the response profile evaluation device 1 according to the embodiment has: the data sampling unit 56 resampling the rating value data 81 to generate the balanced rating value data 85 in which the kinds of the rating value data or the number of pieces of the rating value data is artificially balanced; and the unit 57 for evaluating an RP distribution with a confidence interval, estimating an RP distribution on the basis of a plurality of pieces of balanced rating value data 85 generated and calculating the RP distribution data 86 with a confidence interval for a group of the RP distribution data obtained from the balanced rating value data 85.

With the configuration, the response profile evaluation device 1 according to the embodiment can estimate the RP of the user in a more plausible manner while artificially securing the content independence even in the case where a tendency occurs in the number of response times or the kinds of responses. Further, by estimating an RP distribution for a plurality of pieces of the balanced rating value data 85, the degree of fluctuation which may occur in the RP distribution of the user and a confidence interval can be estimated, and an effect that the reliability of the estimated RP distribution can be considered is obtained.

### Third Embodiment

A response profile evaluation device, a response profile estimation method, and a response profile evaluation program according to a third embodiment of the present invention will be described. Hereinafter, the third embodiment will be described mainly with respect to points different from the first and second embodiments.

The third embodiment relates to an example in which an RP in an intersubject analysis using a psychological measurement value can be considered by using an estimated RP. In the embodiment, an example of receiving the rating value data 81 obtained by measuring the subjective health condition of the user as a psychological measurement value and detecting a change in the subjective health condition of the user will be described.

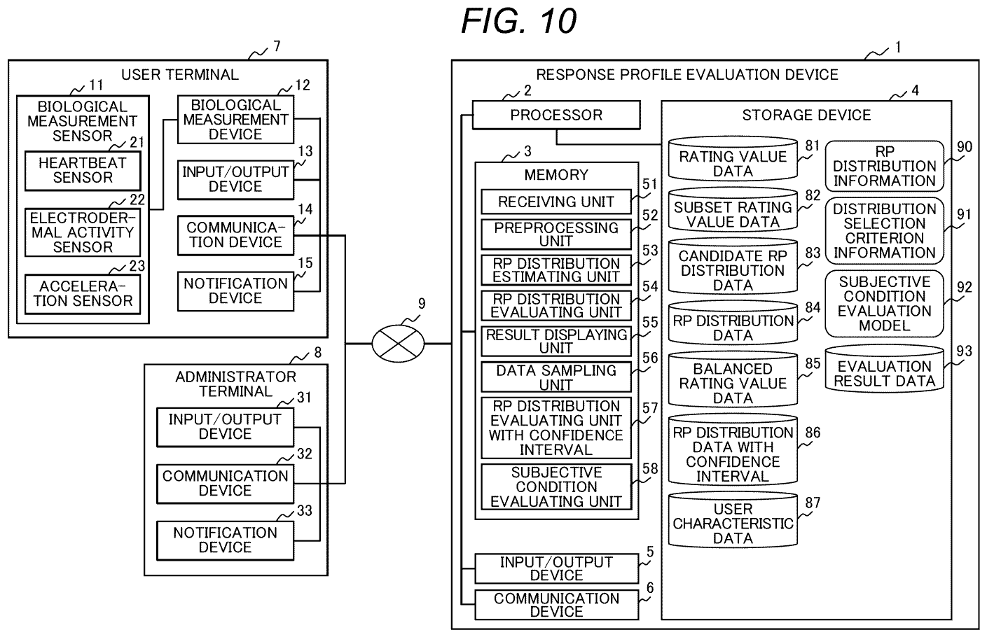

FIG. 10 is a block diagram illustrating an example of main components of the response profile evaluation device 1 according to the embodiment. The response profile evaluation device 1 according to the third embodiment has a subjective condition evaluating unit 58 and a subjective condition evaluation model 92 in the response profile evaluation device (FIG. 1) in the first and second embodiments, and stores evaluation result data 93.

Hereinafter, as an example of detecting a change in the subjective health condition of the user, an example of performing anomaly detection by making comparison with the rating value data 81 measured in the past and determining whether abnormality occurs in the tendency of the rating value data 81 in a predetermined latest period will be described.

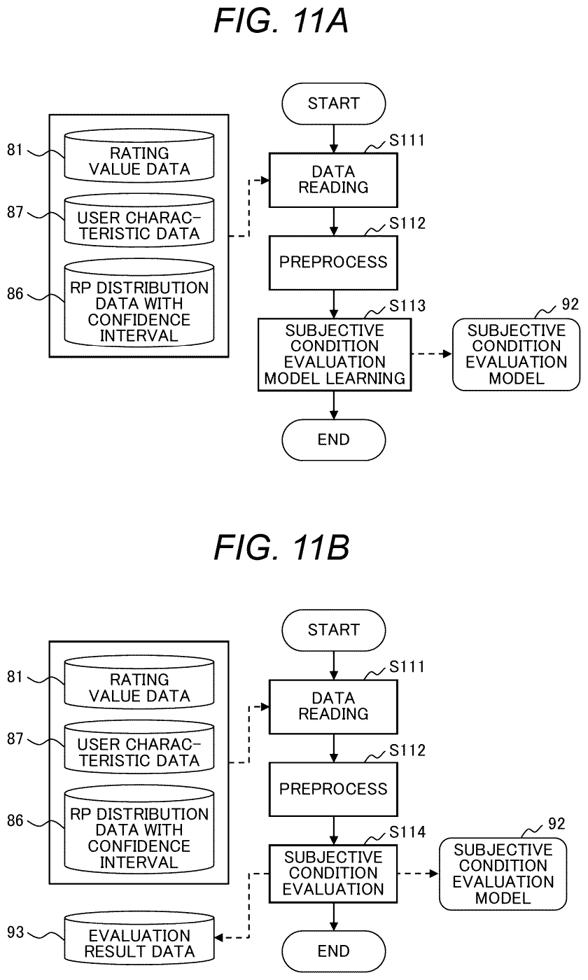

FIG. 11A is a flowchart illustrating an example of processes of leaning the subjective condition evaluation model 92, which are performed by the response profile evaluation device 1. The subjective condition evaluation model 92 is learned according to the flowchart of FIG. 11A.

In a data reading process S111, the receiving unit 51 receives the rating value data 81, the user characteristic data 87, and the RP distribution data 86 with a confidence interval. Although the RP distribution data 86 with a confidence interval is received as an example in the embodiment, in place of the RP distribution data 86 with a confidence interval, the RP distribution data 84 may be received.

In a preprocessing S112, the preprocessing unit 52 preprocesses the data received in the data receiving process S111.

First, the preprocessing unit 52 performs a process corresponding to the preprocess illustrated in FIG. 3 on the rating value data 81.

Subsequently, the preprocessing unit 52 generates a training dataset from the data received in the data reading process S111. For example, in the case where the predetermined latest period is set to one week and anxiety in daily life as the subjective health condition of the user is measured as the rating value data 81, for a period in which abnormality does not occur in anxiety, a data set made by the rating value data 81 of the anxiety of one week, the user characteristic data 87, and the RP distribution data 86 with a confidence interval is cut by a moving window on a day-to-day basis to generate a training dataset. At this time, since the RP of the user has a response tendency which is relatively stable also with time regardless of an instruction, it is considered as a static characteristic, and the RP distribution data 86 with a confidence interval which was evaluated before may be used. On the other hand, in a situation that the characteristic of a change with time of the RP itself has to be considered, for example, the RP distribution data 86 with a confidence interval of each day is calculated and, like the rating value data 81 of anxiety, the RP distribution data 86 with a confidence interval of one week may be cut out by a moving window on a day-to-day basis and added to a data set.

In a subjective condition evaluation model learning process S113, the subjective condition evaluating unit 58 generates the subjective condition evaluation model 92 by using the generated training dataset. The subjective condition evaluation model 92 is a model for receiving a data set made by the rating value data 81 of anxiety of one week received, the user characteristic data 87, and the RP distribution data 86 with a confidence interval, and evaluating the degree of deviation of the data set as compared with that in normal time as the degree of abnormality.

The subjective condition evaluation model 92 is typically an unsupervised model based on a known anomaly detection algorithm and can be configured as a known statistical model or a machine learning model. As the statistical model, for example, a statistical model outputting z-score by using a mean or a standard deviation for the data distribution of the data set, a statistical model outputting a deviation score, a statistical model outputting a quantile on a data distribution, or the like can be used. As the machine learning model, for example, a machine learning model outputting a distance using, as a reference, the cluster center of a data distribution estimated nonparametrically from the data set can be used.

In the case of an unsupervised model based on the anomaly detection algorithm as the subjective condition evaluation model 92 of the user, simply, it is sufficient to generate a model for each user. In this case, as an example of using the RP of the method for an intrasubject analysis, by using the RP distribution data 86 with a confidence interval in addition to the rating value data 81 as an input, a subjective condition can be evaluated also in consideration of the time change characteristic of the RP. An effect that changes in the subjective health condition can be detected with high detection precision by separating the changes into a change in the RP and a change in the rating value data 81.

On the other hand, in the case of generating a model for each user, the large amount of the rating value data 81 of a specific user is necessary. Since responses of the user are indispensable for the rating value data 81, the measurement cost is high. In the case of simply collecting a plurality of users and generating the subjective condition evaluation model 92, a number of pieces of the rating value data 81 can be used as a training dataset. However, since the RP varies among the users, abnormality is excessively detected for a user whose RP is different from the other users. In the method, when the RP distribution data 86 with a confidence interval is used for an input, as an example of using the RP of the method for an intersubject analysis, the RP characteristic can be also considered as an input data set. Consequently, by collecting a plurality of users to generate the subjective condition evaluation model 92, a large amount of data for learning can be used and RPs different among the users are also considered, and an effect that a change in the subjective health condition can be detected with high detection precision is obtained.

The series of learning processes illustrated in FIG. 11A are executed at least once before a subjective condition evaluating process (FIG. 11B) which will be described later is performed. By executing the learning process every predetermined period as the rating value data 81 increases, the subjective condition evaluation model 92 can be re-trained. In such a manner, the subjective condition evaluation model 92 with higher evaluation precision can be generated.

FIG. 11B is a diagram illustrating an example of processes of subjective condition evaluation, which are performed by the response profile evaluation device 1. The response profile evaluation device 1 evaluates the subjective health condition of the user by using the subjective condition evaluation model 92.

In the data reading process S111, the receiving unit 51 receives the rating value data 81, the user characteristic data 87, and the RP distribution data 86 with a confidence interval.

In the preprocessing S112, the preprocessing unit 52 generates an input data set for evaluating a subjective condition. For example, the preprocessing unit 52 generates, as an input data set, a data set made by the rating value data 81 of anxiety of latest one week of the user, the user characteristic data 87, and the RP distribution data 86 with a confidence interval.

In a subjective condition evaluation S114, the subjective condition evaluating unit 58 evaluates the subjective health condition of the user by using an input data set generated by the preprocess S112 and the subjective condition evaluation model 92, and obtains the degree of abnormality indicating whether a change of the subjective health condition from usual condition is remarkable or not. The subjective condition evaluating unit 58 outputs the obtained degree of abnormality as, for example, continuous values from 0 to 1, and stores it in the evaluation result data 93.

Consequently, the response profile evaluation device 1 of the embodiment has an effect that a change in the subjective health condition of the user can be detected with high precision in consideration of RPs which are different among users.

### Fourth Embodiment

A response profile evaluation device, a response profile evaluation method, and a response profile evaluation program of a fourth embodiment of the present invention will be described. Hereinafter, the fourth embodiment will be described mainly with respect to the points different from the first and second embodiments.

In the third embodiment, the rating value data 81 itself is input as a target to be analyzed, and a change in the subjective health condition of the user is detected in consideration of the RP of the user.

In the fourth embodiment, in the case of receiving biological measurement data as data obtained by measuring a living body (user) by the biological measurement sensor 11 and estimating the rating value data 81, the subjective health condition of the user is estimated in consideration of the RP of the user. In the embodiment, an example of setting the subjective health condition to be estimated as natural emotion in daily life, estimating the natural emotion measured in a feeling dimension made by arousal and valence from the biological measurement data and, in the case of feeling which does not often arise in daily life, requesting a response as the rating value data 81 will be described.

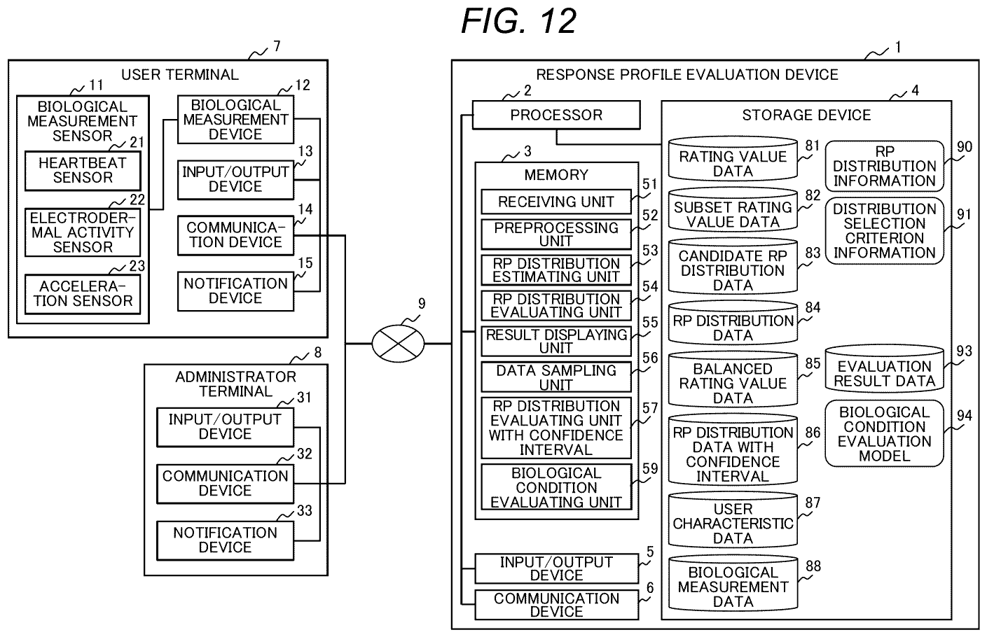

FIG. 12 is a block diagram illustrating an example of main components of the response profile evaluation device 1 according to the fourth embodiment. The response profile evaluation device 1 of the fourth embodiment has, in the response profile device (FIG. 1) of the first and second embodiments, a biological condition evaluating unit 59, biological measurement data 88, and a biological condition evaluation model 94, and stores the evaluation result data 93. The biological condition evaluation model 94 is a model for estimating the subjective health condition of the user from the biological condition of the user.

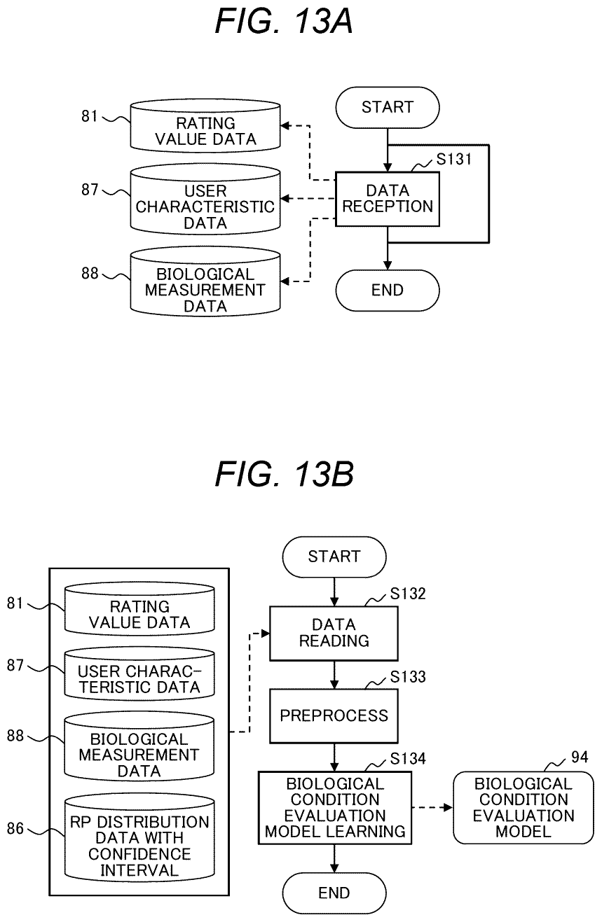

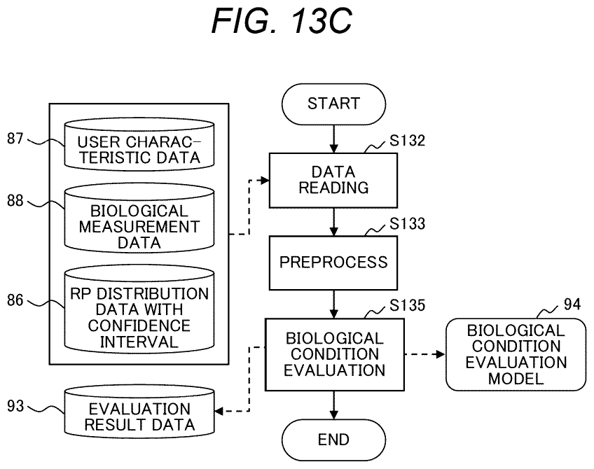

FIG. 13A is a flowchart illustrating an example of processes of receiving data from the user terminal 7 by the response profile evaluation device 1. In a data receiving process S131, the receiving unit 51 of the response profile evaluation device 1 receives the rating value data 81 and the user characteristic data 87 from the user terminal 7 in a manner similar to the data receiving process S21 in FIG. 2 (first embodiment), and also receives the biological measurement data 88 from the user terminal 7.

FIG. 13B is a flowchart illustrating an example of learning process of the biological condition evaluation model 94 performed by the response profile evaluation device 1. The biological condition evaluation model 94 is learned according to the flowchart of FIG. 13B.

In a data reading process S132, the receiving unit 51 receives the rating value data 81, the user characteristic data 87, the biological measurement data 88, and the RP distribution data 86 with a confidence interval. Although the RP distribution data 86 with a confidence interval is received in the embodiment as an example, in place of the RP distribution data 86 with a confidence interval, the RP distribution data 84 may be received.

Hereinafter, an example will be described in which, unless otherwise described, a set of the biological measurement data 88 is made by R-R interval data, electrodermal activity data, and acceleration data in thirty minutes, a set of the rating value data 81 is the intensity of valence and arousal in the 30 minutes and, by using a plurality of the sets, the biological condition evaluation model 94 is learned. Therefore, the rating value data 81 corresponds to correct answer data for estimating the subjective health condition.

In a preprocess S133, the preprocessing unit 52 preprocesses input data. First, the preprocessing unit 52 performs a process corresponding to the preprocess S32 in FIG. 3 (first embodiment) on the rating value data 81 as correct answer data for estimating the subjective health condition.

Except for this process, for example, the preprocessing unit 52 may convert the rating value data 81 to a quantile for each user on the basis of the RP distribution data 86 with a confidence interval. The quantile conversion is a process of converting data to probability of occurrence or the like on the basis of cumulative density function of the occurrence frequency. For example, in the case of using the normal distribution approximation method as the RP distribution data 86 with a confidence interval, a cumulative density function of an RP distribution is obtained by using a mean of a bootstrap distribution, and relative occurrence probability (a value from 0 to 1) of the rating value data 81 for each user may be used as correct answer data.

In the case of classifying natural emotion in place of estimation of continuous values of natural emotion as the subjective health condition, binarization may be performed by, for example, using a margin ε, eliminating data in the range of 0.5+ε as the value of median from 0 to 1, setting the range from 0 to 0.5−ε as a negative case of 0, and setting the range from 0.5+ε to 1 as a positive case of 1.

The preprocessing unit 52 performs, as the preprocess S133 for the biological measurement data 88, at least one of a process of correcting an individual difference included in the biological measurement data 88, a process of extracting a feature from the biological measurement data 88, and a process of compressing the characteristic extracted from the biological measurement data 88 (dimension reduction). With respect to those processes, the preprocessing unit 52 may explicitly sequentially execute a plurality of processes (subtasks) as a pipeline or execute the processes in an end-to-end manner (at once without dividing the task into subtasks).

For example, a series of preprocesses S133 may be configured by performing a process of normalizing data based on the series of biological measurement data 88 measured from the user himself/herself in the past and performing a process of compressing a characteristic by a principal component analysis using the biological measurement data 88 normalized for each user as an input without performing the feature extracting process.

In the case of performing the processes in the end-to-end manner, the preprocess S133 may be configured in a manner that a process of eliminating measurement noise of the biological measurement data 88 is set and, as the feature extracting process and the dimension reduction process for features, a latent vector of a variational auto encoder (VAE) as a deep generative model using the biological measurement data 88 from which noise is eliminated as an input is used. In this case, a VAE model is learned by unsupervised learning from the biological measurement data 88.

For the purpose of individual difference correction or noise reduction, a process of normalizing a signal scale used and a noise eliminating process may be performed. In the normalizing process for example, for each user, every measurement date, every measurement day of the user, or among all of the users, min-max normalization of performing normalization with the maximum value and the minimum value, z-score process of performing normalization by an average of signals and a standard deviation, a quantile normalization of performing normalization by using a quantile in a signal intensity distribution, or the like may be used. In the case where it is known that signal intensity fluctuates due to aging or the like, a deviation scoring process for normalizing the signal intensity for each of age groups in consideration of information of the age of the user included in the user characteristic data 87 may be performed. Further, in the noise eliminating process, a clipping process or winsorize process for making values lie in a predetermined range by eliminating outlier of signals, a moving average process for performing smoothing by suppressing temporal abrupt fluctuations, a zero-order differential process with a Savizky-Golay filter, or the like may be performed.

In the process of extracting a feature from the biological measurement data 88, a feature may be extracted according to a biological signal of the biological measurement data 88 used. For example, for R-R interval data obtained by the heartbeat sensor 21, an average heart rate, a low frequency component (LF) and a high frequency component (HF) which are known to be obtained by frequency domain analysis and reflecting mainly sympathetic nerve activity and parasympathetic nerve activity, SDNN, RMSSD, and NN50 used in time domain analysis, features using Lorentz plots (Poincare plot) used in nonlinear domain analysis, features obtained by the detrended fluctuation analysis, features obtained by the complex demodulation method, or the like can be used. For electrodermal activity data obtained by the electrodermal activity sensor 22, skin conductance level (SCL) or skin conductance response (SCR) may be used. For three-dimensional acceleration data obtained by the acceleration sensor 23, an acceleration norm, the number of zero-crossings a signal processed by a band-pass filter for an acceleration norm crosses the threshold of ±0.01G when gravity acceleration is 1G, or the like may be used.

For a dimension reduction process for features, a known algorithm can be used. For example, the above-described principal component analysis, an auto encoder (AE), uniform manifold approximation and projection (UMAP), or the like may be used.

After that, the preprocessing unit 52 generates a training dataset by using the data subjected to the various converting processes. For example, a training dataset may be generated by setting the biological measurement data 88, the RP distribution data 86 with a confidence interval, and the user characteristic data 87 as an input X of the biological condition evaluation model 94, and setting the rating value data 81 as an output y of the biological condition evaluation model 94. Alternatively, a training dataset may be generated by setting the biological measurement data 88 and the user characteristic data 87 subjected to the converting process as an input X and setting the rating value data 81 subjected to quantile conversion by the RP distribution data 86 with a confidence interval as an output y. Moreover, a training dataset may be generated by combining the processes.

After completion of the series of preprocesses S133, a biological condition evaluation model learning process S134 is performed.

In the biological condition evaluation model learning process S134, the biological condition evaluating unit 59 generates the biological condition evaluation model 94 by using the generated training dataset.

For example, when the biological condition evaluation model 94 is a model using natural emotion in daily life of the user as an object to be estimated, the biological condition evaluation model 94 may learn a feeling dimension estimation model which estimates “intensity of valence and arousal in certain 30 minutes” as natural emotion by supervised learning. The biological condition evaluation model 94 can be generated by using a known algorithm. For example, for a machine learning algorithm, a logistic regression model, decision tree, random forest, support vector machine, neural network, Bayesian neural network, deep learning model, or the like can be used. As the algorithm, a classification algorithm or a regression algorithm can be also used according to the subjective health condition to be estimated. For example, in the case of estimating the intensity of valence and arousal by a value from 0 to 1, a regression algorithm can be used. In the case of estimating the level (high/low) of the intensity of valence and arousal, a classification algorithm may be used in place of the regression algorithm.

Further, the biological condition evaluation model 94 may be configured by a plurality of models in which differences among individuals are considered. In this case, the subjective health condition adapted to each user can be estimated, and an effect that fitness to the user and acceptability of a result of subjective value estimation of the user improves is obtained.

In the case of configuring the biological condition evaluation model 94 by a plurality of models in which differences among individuals are considered, a dedicated process may be added to the biological condition evaluation model 94, or the biological condition evaluation model 94 itself may be configured in an end-to-end form. For example, the case of setting the rating value data 81 which is subjected to the quantile conversion by the RP distribution data 86 with a confidence interval as the output y corresponds to the example of adding a dedicated process. In the case of configurating an end-to-end form, by configuring the biological condition evaluation model 94 by a neural network, and configuring a layer close to the final layer by multitask learning of dividing the layer for each user or a group of users whose RP distributions are close, estimation according to the RP of the user can be realized. It is also possible to configure so that, by generating a biological condition evaluation model 94 common to the users and finely tuning the biological condition evaluation model 94 to each of the users, the biological condition evaluation model 94 is fit to the user having a specific RP.

A series of learning processes in FIG. 13B are executed at least once before a biological condition evaluating process (FIG. 13C) which will be described later is performed. By performing the learning process every predetermined time as the rating value data 81 and the biological measurement data 88 increases, the biological condition evaluation model 94 can be re-trained. By the above, the biological condition evaluation model 94 with higher evaluation precision can be generated.

FIG. 13C is a diagram illustrating an example of biological condition evaluating process performed by the response profile evaluation device 1. The response profile evaluation device 1 evaluates the subjective health condition of the user from the biological condition of the user by using the biological condition evaluation model 94.

In the data reading process S132, the receiving unit 51 receives the user characteristic data 87, the biological measurement data 88, and the RP distribution data 86 with a confidence interval.

In the preprocess S133, the preprocessing unit 52 generates an input data set for evaluating the subjective health condition of the user from the biological condition of the user. For example, the preprocessing unit 52 generates, as an input data set, a data set made by the user characteristic data 87, the biological measurement data 88, and the RP distribution data 86 with a confidence interval.

In a biological condition evaluating process S135, the biological condition evaluating unit 59 estimates the subjective health condition of the user by using the input data set generated in the preprocess S133 and the biological condition evaluation model 94. For example, in the case of estimating “the intensity of valence and arousal in certain 30 minutes”, the estimated intensity of arousal and valence is estimated as continuous values from 0 to 1. The biological condition evaluating unit 59 outputs the subjective health condition estimated and obtained and stores it in the evaluation result data 93.

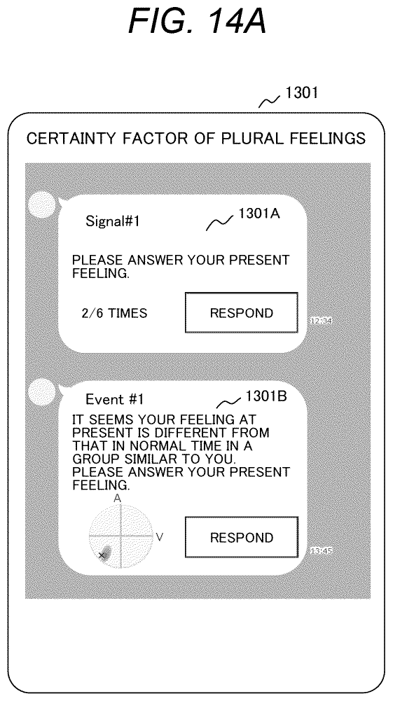

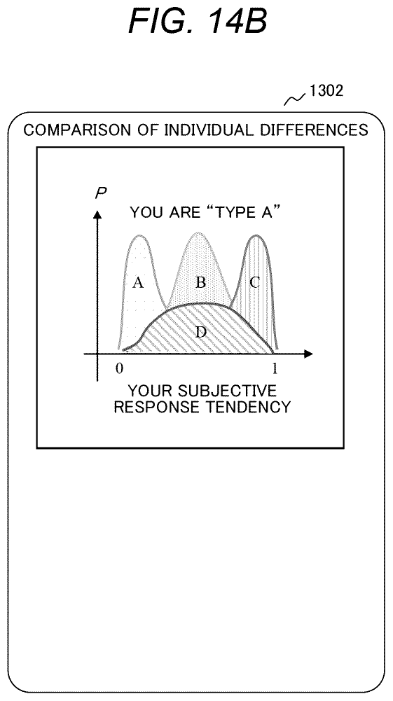

FIGS. 14A and 14B are diagrams illustrating an example of an evaluation result which is output and displayed on the display of the input/output device 13 of the user terminal 7 by the result display unit 55. FIGS. 14A and 14B illustrate the case of using a smartphone as the input/output device 13 as an example. In the embodiment, an example of the case where an evaluation result is notified to the user in the form of a message to an application for notification will be described.

A screen 1301 in FIG. 14A is an example of the display screen for requesting the user to enter the rating value data 81 (a response to an instruction) on the basis of the evaluation result data 93.

In the conventional technique, for example, an application for notification irregularly displays a notification 1301A to the user like Signal #1 on the screen 1301 of FIG. 14A and tries to collect the rating value data 81 as correct answer data of feeling at a certain moment. However, feeling such as strong joy or sadness does not arise frequently in daily life. Consequently, simple irregular notifications result in long time to collect correct answer data or termination of a predetermined period without acquiring sufficient number of pieces of correct answer data.

In the embodiment, since the evaluation result data 93 is obtained in consideration of the RP of the user, whether or not the user is in condition different from that in normal time can be evaluated in consideration of the RP. Consequently, for example, when it is assumed that the state where the estimation intensity of arousal and valence is 0 or close to 1 corresponds to a strong feeling, a timing when the user is in the subjective condition (feeling state) different from that in normal time can be displayed to the user terminal 7 by a notification 1301B of a form of Event #1 illustrated in the screen 1301. It can move the user to respond when it is estimated that a feeling arising in the user is not that in daily life. As a result, it can contribute to collection of the rating value data 81.

In the embodiment, in such a manner, a response of the user based on the notification 1301B can be obtained as the rating value data 81 in the data receiving process S131 in FIG. 13A. Consequently, in the embodiment, an effect is obtained such that the rating value data 81 corresponding to a rare feeling state can be obtained with high efficiency as compared with the conventional technique of irregularly displaying the notification 1301A such as Signal #1 to the user to obtain rating value data, and using the rating value data as correct answer data of feeling.

A screen 1302 of FIG. 14B is an example of a display screen illustrating to the user an example of a plurality of types A to D of the RP and the type of the RP of the user. The screen 1302 indicates the type of the RP of the user in the case of generating a plurality of models in which differences of individuals are considered for the respective types A to D (a group of similar users A to D) of the RP by using the RP distribution data 86 with a confidence interval. By the display screen such as the screen 1302, the user can make comparison with other users or the group of similar users different from himself/herself and determine the response characteristic (that is, RP) of himself/herself.

Consequently, the response profile evaluation device 1 of the embodiment has an effect that the subjective health condition of the user can be estimated with high precision from the biological measurement data 88 in consideration of an RP which varies among users.

The present invention is not limited to the foregoing embodiments but can be variously modified. For example, the foregoing embodiments have been specifically described so that the present invention is easily understood. The present invention is not always limited to a mode having all of the components described. A part of the configuration of an embodiment can be replaced with the configuration of another embodiment. To the configuration of an embodiment, the configuration of another embodiment can be added. A part of the configuration of each of embodiments can be eliminated, or another configuration can be added or replaced.

The components of the device according to the present invention may be realized by hardware by, for example, designing a part or all of the components by an integrated circuit. A part or all of the components may be realized by software so that the processor 2 interrupts and executes a program realizing the functions. A program, a table, a file, measurement information, and information such as calculation information realizing the functions can be recorded in a recording device such as the memory 3, a hard disk drive, and SSD (Solid State Drive), and a computer-readable recording medium such as an IC card, an SD card, a DVD, or the like. Therefore, the components of the device according to the present invention as a processor, a processing unit, a program module, and the like can realize the respective functions.

In each of the diagrams, the control lines and information lines considered to be necessary for the description are drawn. All of the control lines and information lines necessary as a product are not always drawn. It can be considered that almost all of the components are mutually connected in an actual product.

### REFERENCE SIGNS LIST

1: response profile evaluation device
2: processor
3: memory
4: storage device
5: input/output device
6: communication device
7: user terminal
8: administrator terminal
9: network
11: biological measurement sensor
12: biological measurement device
13: input/output device
14: communication device
15: notification device
21: heartbeat sensor
22: electrodermal activity sensor
23: acceleration sensor
31: input/output device
32: communication device
33: notification device
51: receiving unit
52: preprocessing unit
53: RP distribution estimating unit
54: RP distribution evaluating unit
55: result displaying unit
56: data sampling unit
57: unit for evaluating RP distribution with confidence interval
58: subjective condition evaluating unit
59: biological condition evaluating unit
81: rating value data
82: subset rating value data
83: candidate RP distribution data
84: RP distribution data
85: balanced rating value data
86: RP distribution data with confidence interval
87: user characteristic data
88: biological measurement data
90: RP distribution information
91: distribution selection criterion information
92: subjective condition evaluation model
93: evaluation result data
94: biological condition evaluation model
501: table
502A, 502B: graph
601A, 601B: graph
901A: histogram
901B: probability density function
1301: screen
1301A: notification
1301B: notification
1302: screen
7101: user ID
7102: process date
7103: process ID
7104: data start date/time
7105: data end date/time
7106: RP distribution information ID
7107: distribution selection criterion information ID
7108: RP distribution parameter
7109: DistSub

What is claimed is:

1. A response profile evaluation device, using a response tendency of a user as a response profile, and using a statistical distribution expressing the response profile of the user as a response profile distribution,
the response profile evaluation device comprising:
a receiving unit for receiving rating value data of the user;
a response profile distribution estimating unit for estimating, on the basis of the rating value data, a plurality of response profile distribution candidates that are candidates of the response profile distribution, by using a candidate response profile distribution template including a type of the statistical distribution; and
a response profile distribution evaluating unit for selecting the response profile distribution that is fitted to expression of the response profile of the user from the plurality of estimated response profile distribution candidates by using the rating value data.

2. The response profile evaluation device according to claim 1,
wherein the response profile distribution estimating unit estimates a plurality of response profile distribution candidates including at least one of a main response profile distribution candidate and a sub response profile distribution candidate, the main response profile distribution candidate expressing the response profile appearing in neighborhood of center of a domain of the rating value data, and the sub response profile distribution candidate expressing the response profile appearing at least one of neighborhood of an upper limit and neighborhood of a lower limit of the domain of the rating value data, and
wherein the response profile distribution evaluating unit selects one response profile distribution, a plurality of response profile distributions, or a combination of a plurality of response profile distributions, that are fitted to expression of the response profile of the user from the plurality of estimated response profile distribution candidates.

3. The response profile evaluation device according to claim 2, further comprising:
a data sampling unit that resamples the rating value data and generates balanced rating value data in which kinds of the rating value data or number of pieces of the rating value data are balanced in an artificial-manner; and
a unit of evaluating a response profile distribution with a confidence interval, which calculates a response profile distribution with a confidence interval that is the response profile distribution with a plausible range,
wherein the response profile distribution estimating unit estimates the response profile distribution candidate on the basis of the plurality of pieces of balanced rating value data,
wherein the response profile distribution evaluating unit selects the response profile distribution by using the plurality of pieces of balanced rating value data, and
wherein the unit of evaluating a response profile distribution with a confidence interval calculates the response profile distribution with a confidence interval from the selected response profile distribution.

4. The response profile evaluation device according to claim 1, further comprising:
a result display unit that displays at least one of the response profile distribution and a parameter group that characterizes the response profile distribution.

5. The response profile evaluation device according to claim 2, further comprising:
a preprocessing unit that divides the rating value data by a data range of the domain to generate subset rating value data for estimating the main response profile distribution candidate and the sub response profile distribution candidate.

6. The response profile evaluation device according to claim 1,
wherein the response profile distribution is expressed by a distribution made by a single statistical distribution or a mixture distribution of a plurality of statistical distributions and is expressed by a parameter group that characterizes the distribution.

7. The response profile evaluation device according to claim 1,
wherein the response profile distribution evaluating unit selects the response profile distribution by using, as an evaluation index, at least one of a likelihood related statistic, an information criterion, a distribution similarity, and the degree of separation of peaks of a mixture distribution.

8. The response profile evaluation device according to 1, further comprising:
a condition evaluating unit that evaluates condition of the user by using the response profile distribution and at least one of the rating value data and biological measurement data obtained by measuring a biological condition of the user.

9. The response profile evaluation device according to claim 1,
wherein the rating value data is data measured by using a measurement scale of a continuous response type.

10. A response profile evaluation method executed by a computer comprising a receiving unit, a response profile distribution estimating unit, and a response profile distribution evaluating unit, and using a response tendency of a user as a response profile, and using a statistical distribution expressing the response profile of the user as a response profile distribution, comprising:
a step by the receiving unit, of receiving rating value data of the user;
a step by the response profile distribution estimating unit, of estimating, on the basis of the rating value data, a plurality of response profile distribution candidates that are candidates of the response profile distribution, by using a candidate response profile distribution template including a type of the statistical distribution; and
a step by the response profile distribution evaluating unit, of selecting the response profile distribution that is fitted to expression of the response profile of the user from the plurality of estimated response profile distribution candidates by using the rating value data.

11. A response profile evaluation program for making a computer have functions of the receiving unit, the response profile distribution estimating unit, and the response profile distribution evaluating unit of the response profile evaluation device according to claim 1.
